(19) United States
(12) Patent Application Publication
(10) Pub. No.: US 2026/0199086 A1
(43) Pub. Date: Jul. 16, 2026

(54) PROSTHETIC HEART VALVES AND METHODS FOR SUPRA-ANNULAR IMPLANTATION

(71) Applicant: Edwards Lifesciences Corporation, Irvine, CA (US)

(72) Inventors: Nikolai Gurovich, Hadera (IL); Tamir S. Levi, Zikhron Yaakov (IL)

(73) Assignee: Edwards Lifesciences Corporation, Irvine, CA (US)

(21) Appl. No.: 19/564,000

(22) Filed: Mar. 11, 2026

Related U.S. Application Data
(63) Continuation of application No. PCT/US2024/048174, filed on Sep. 24, 2024.
(60) Provisional application No. 63/540,240, filed on Sep. 25, 2023.

(51) Int. Cl. A61F 2/24

(52) U.S. Cl. CPC A61F 2/2418; A61F 2/2436; A61F 2220/0008

(57) ABSTRACT

Prosthetic heart valves, delivery apparatus, and methods for preventing or limiting migration of an implanted prosthetic valve. The prosthetic valves can be configured to be implanted between an annulus and a sinotubular junction (STJ) of a native aortic valve. The prosthetic valve in a radially expanded state can have a height that is less than a distance between the annulus and the STJ. The prosthetic valve can additionally include one or more anti-migration features. A method includes implanting a prosthetic valve within the native aortic valve such that an inflow end of a frame of the radially expanded prosthetic valve is above the annulus and an outflow end of the frame of the radially expanded prosthetic valve is below the STJ.

### CROSS-REFERENCE TO RELATED APPLICATIONS

This application is a continuation of PCT Application No. PCT/US2024/048174, filed September 24, 2024, which claims the benefit of U.S. Provisional Patent Application No. 63/540,240, filed September 25, 2023, the entire contents of each of which are incorporated herein by reference.

### FIELD

The present disclosure relates to prosthetic heart valves, and in particular to prosthetic heart valves for supra-annular implantation and associated methods of implantation.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

When implanted in a native aortic valve, the deployed, radially expanded prosthetic heart valve is normally positioned such that an inflow end portion of the prosthetic valve is seated within or below the aortic annulus. For example, 20-30% of the length of the prosthetic valve can extend below the aortic annulus and into the left ventricle (for example, into the left ventricular outflow tract (LVOT)), while the remainder of the prosthetic valve is seated within and/or above the annulus. In some examples, a prosthetic valve can have a longer length that results in an outflow end of a prosthetic valve extending into the ascending aorta, above a sinotubular junction (STJ) between the aortic valve and the ascending aorta.

### SUMMARY

Described herein are prosthetic heart valves, delivery apparatus, and methods for implanting prosthetic heart valves. The disclosed prosthetic heart valves, delivery apparatus, and methods can, for example, prevent or limit migration of an implanted prosthetic valve and/or can reduce a force exerted on a native annulus by an implanted prosthetic valve. For example, the methods described herein include implanting a prosthetic valve within a native aortic valve above the aortic annulus and below the sinotubular junction (STJ). As such, the devices and methods disclosed herein can, among other things, overcome one or more of the deficiencies of typical prosthetic heart valves and their associated methods of implantation.

A prosthetic heart valve can comprise a frame and a valve structure coupled to the frame. In addition to these components, a prosthetic heart valve can further comprise one or more of the components disclosed herein.

In some examples, the prosthetic heart valve in a radially expanded state can have a height that is less than a distance between a native aortic annulus and a native STJ of a patient.

In some examples, the prosthetic heart valve in a radially expanded state can have a height in a range of 15.5 mm to 22.5 mm.

In some examples, the prosthetic heart valve can be configured to be seated or implanted between the native aortic annulus and the native STJ.

In some examples, the native leaflets can extend along an exterior surface of the prosthetic heart valve when the prosthetic heart valve is implanted.

In some examples, an inflow end of the implanted prosthetic heart valve is configured to abut or otherwise engage a base portion of the native leaflets.

In some examples, an outflow end of the implanted prosthetic heart valve is configured to abut or otherwise engage a top wall of the aortic valve below the STJ.

In some examples, the prosthetic heart valve can include one or more anti-migration features.

In some examples, the anti-migration features can include clipping members.

In some examples, the anti-migration features can include an outer skirt.

In some examples, the outer skirt can include an annular extension that extends into or below the aortic annulus to prevent or limit tissue ingrowth.

In some examples, the outer skirt can comprises a radially extending flange.

A method of implanting a prosthetic heart valve can comprise inserting a distal end portion of a delivery apparatus having the prosthetic heart valve mounted thereon in a radially compressed state into a vasculature of a patient, advancing the distal end portion of the delivery apparatus through the vasculature until the radially compressed prosthetic heart valve is within a native aortic valve of the patient, and radially expanding the prosthetic heart valve to a radially expanded state within the native aortic valve, wherein an inflow end of the radially expanded prosthetic heart valve is above an annulus of the aortic valve and an outflow end of the radially expanded prosthetic heart valve is below an STJ.

In some examples, the method can include aligning the radially compressed prosthetic heart valve relative to one or more anatomical features of the native aortic valve.

In some examples, the aligning the radially compressed prosthetic heart valve relative to the one or more anatomical features of the native aortic valve can include positioning the outflow end of the prosthetic heart valve above the STJ.

In some examples, the aligning the radially compressed prosthetic heart valve relative to the one or more anatomical features of the native aortic valve can include positioning the inflow end of the prosthetic heart valve below the aortic annulus.

In some examples, radially expanding the prosthetic valve causes the prosthetic valve to foreshorten, which is turn allows the inflow end of the prosthetic valve to move to a location above the aortic annulus and the outflow end of the prosthetic valve to move to a location below the STJ.

In some examples, the method can include deploying one or more anti-migration features.

In some examples, the deploying anti-migration features can include engaging frame anchors such as clipping members that can extend over a free edge of the native leaflets.

In some examples, the inflow end of the radially expanded prosthetic heart valve abuts a base portion of the native leaflets after the prosthetic valve is radially expanded.

In some examples, the outflow end of the radially expanded prosthetic heart valve abuts a top wall of the aortic root below the STJ.

In some examples, the method can include determining a distance between the aortic annulus and the STJ of the patient.

In some examples, the method can include selecting a prosthetic heart valve that has a height in a radially expanded state that is less than the distance between the aortic annulus and the STJ.

The above method(s) can be performed on a living animal or on a simulation, such as on a cadaver, cadaver heart, anthropomorphic ghost, simulator (for example, with body parts, heart, tissue, etc. being simulated).

In some examples, a method comprises one or more of the components recited in Examples 1-37 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to an example.

FIG. 2 is a side view of a frame of the prosthetic heart valve of FIG. 1.

FIG. 3 is a side view of an exemplary delivery apparatus configured to deliver and implant a radially expandable prosthetic heart valve at an implantation site.

FIG. 4 is a cross-sectional view of an exemplary prosthetic heart valve implanted within a native aortic valve.

FIG. 5 is a cross-sectional view of another exemplary prosthetic heart valve implanted within a native aortic valve.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, a deployed, radially expanded prosthetic heart valve is often positioned within a native aortic heart valve such that an inflow end portion of the prosthetic valve is seated within or below the aortic annulus. In some examples, an outflow end portion of the prosthetic valve extends into the ascending aorta above the sinotubular junction (STJ).

Such prosthetic valves and methods of implantation can have various issues associated therewith.

For example, in order to prevent dislodgement of the prosthetic valve, the prosthetic valve exerts a radial force on the native annulus that is sufficient to retain a position of the prosthetic valve. However, if the prosthetic valve is over-expanded, the outward force of the prosthetic valve against the native annulus can cause or pose a risk of annular rupture (also referred to as “aortic root rupture”).

In another example, anchoring of the prosthetic valve within a native annulus can be facilitated by calcification and narrowing of the annulus, which occurs, for example, in patients that have aortic stenosis (AS). However, in other disease conditions, such as aortic insufficiency (AI) (also referred to as “aortic regurgitation”), there may be little to no calcification of the native annulus and engagement between the inflow end of the prosthetic valve and the native annulus may be insufficient to retain a position of the prosthetic valve at the implantation site. Accordingly, there is a risk that the prosthetic valve will migrate downward into the left ventricle.

Similarly, where the prosthetic valve is insufficiently engaged with the native annulus and an outflow end of a prosthetic valve extends into the ascending aorta, there is a risk that the prosthetic valve will migrate upward further into the ascending aorta.

In yet another example, in patients with a high degree of calcification at the native annulus (AS), expansion of the prosthetic valve may cause or pose a risk of injury to the annulus. In yet another example, in patients where the native leaflets are calcified (for example, commissural calcification which may result in fused cusps), it may be necessary to use a smaller prosthetic valve (that is, a smaller prosthetic valve than would normally be selected based on the size of the native annulus), which may have a higher risk of paravalvular leakage (PVL) around the implanted prosthetic valve.

The prosthetic valves and methods disclosed herein can address one or more of the foregoing issues. In some examples, the prosthetic valves described herein are configured to be seated within a native aortic valve above an annulus (supra-annular) and below the ascending aorta, for example, below a sinotubular junction (STJ) (infra-STJ). An inflow end of the prosthetic valve can be configured to be seated above the aortic annulus. For example, the inflow end can contact native leaflet tissue at a base region of the retained leaflets. An outflow end of the prosthetic valve can be configured to be seated below the STJ. For example, the outflow end can contact the wall of the aortic root on an inferior (lower) side of the STJ. In another example, a prosthetic valve can have a length that is less than a distance between a top surface of the annulus and the STJ.

In the foregoing examples, the inflow and outflow ends of the radially expanded prosthetic valve can be abutted to and retained between anatomical structures within the aortic valve (for example, between a top surface of the annulus and the aortic root below the STJ). Accordingly, upstream migration (that is, migration toward the left ventricle) and downstream migration (that is, migration toward the ascending aorta) of the radially expanded prosthetic valve can be prevented or limited via the prosthetic devices and methods of implantation disclosed herein.

Further, exertion of force on the annulus by the radially expanded valve can be prevented or limited via the prosthetic valves and methods of implantation disclosed herein, thereby reducing the risk of injury to the annulus. Additionally, in examples where the native leaflets are calcified and a smaller prosthetic valve is utilized, the prosthetic valve can be implanted such that an outer skirt of the prosthetic valve is positioned at the location of the calcified leaflets and can thereby reduce the likelihood of PVL around the implanted prosthetic valve. In other words, the outer skirt can form a seal with the native leaflets downstream of the native annulus to prevent or minimize paravalvular leakage. In this manner, a prosthetic valve implanted supra-annularly can form a more effective seal for an undersized prosthetic valve compared to an undersized prosthetic valve implanted within the annulus.

In some examples, the prosthetic valves disclosed herein can include additional anti-migration features such as frame anchors (for example, clipping members).

In some examples, the prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

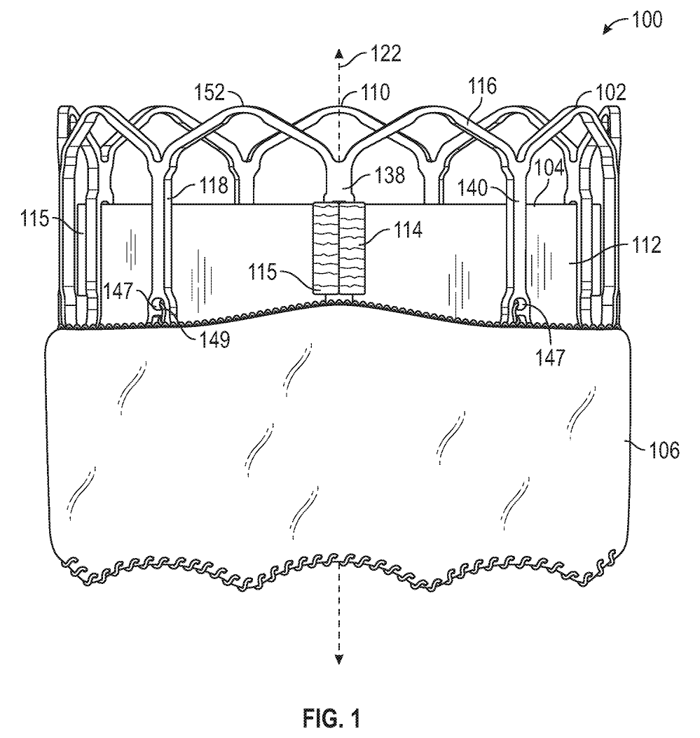

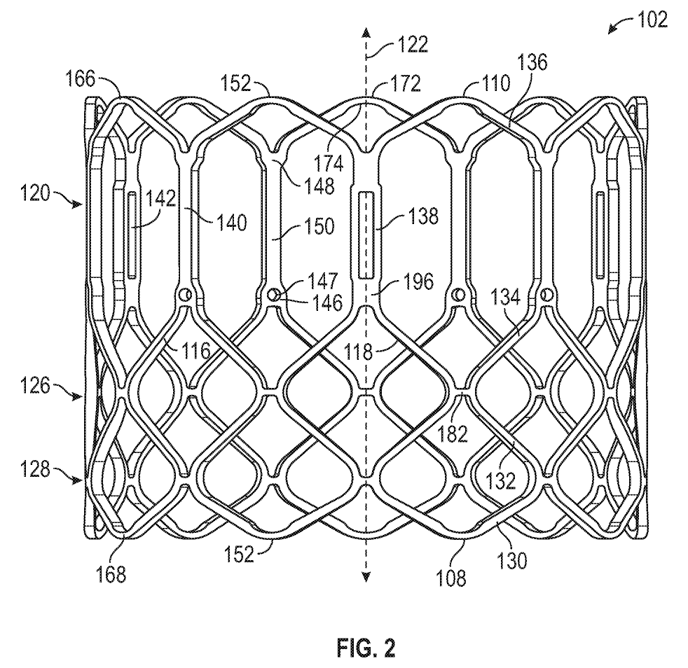

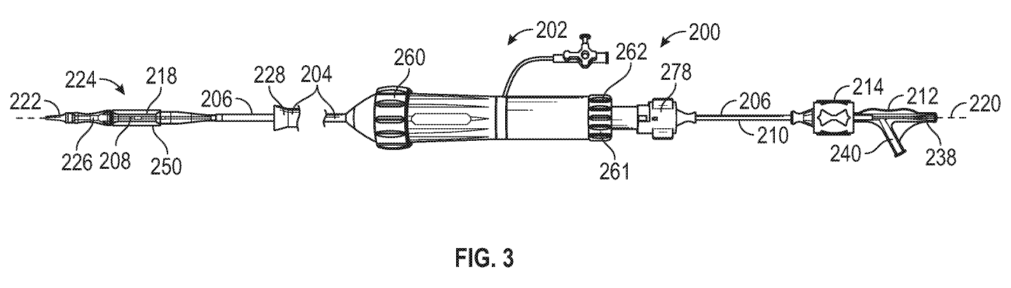

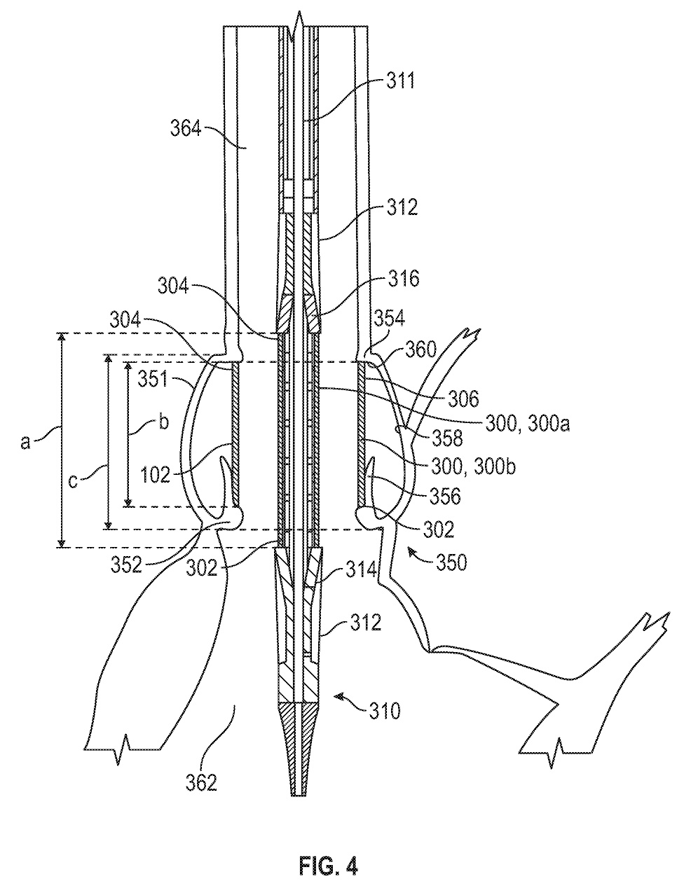

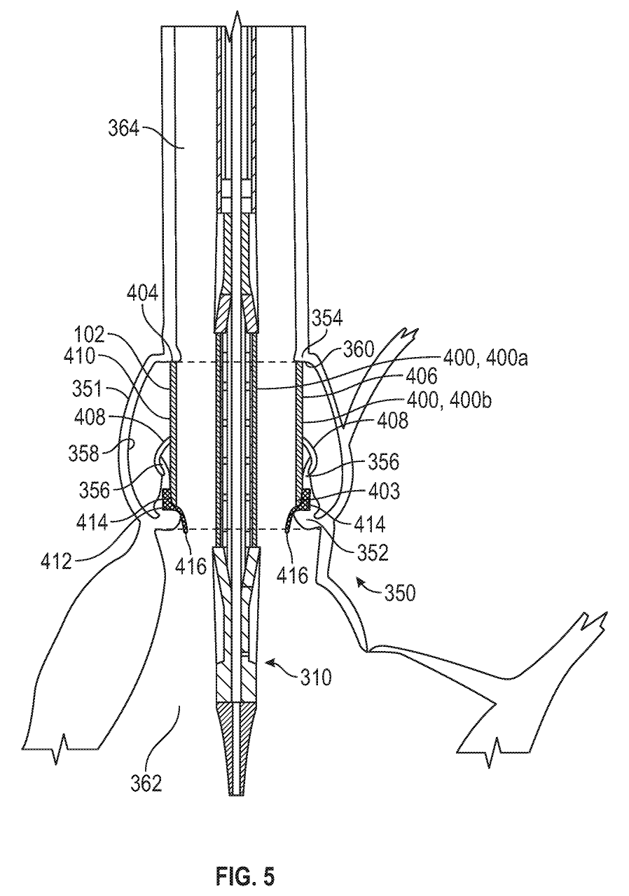

FIG. 1 illustrates an exemplary prosthetic heart valve comprising a frame, leaflets secured on an inside of the frame, and an outer skirt disposed around an outer surface of the frame. In some examples, the frame can comprise a plurality of interconnected and angled struts, including struts that form commissure windows (commissure support portions of the frame) that are configured to receive commissures of the leaflets, as shown in FIGS. 1-2. In some examples, a frame for a prosthetic heart valve can comprise commissure support portions configured to receive commissures of the leaflets, each commissure support portion formed by angled struts forming an open cell of the frame. The prosthetic heart valves can be advanced through a patient’s vasculature, such as to a native heart valve, by a delivery apparatus, such as the exemplary delivery apparatus shown in FIG. 3. FIGS. 4 and 5 illustrate implantation of the exemplary prosthetic heart valves at a native aortic valve, in accordance with the present disclosure.

It will be appreciated that the exemplary prosthetic valves and methods of implantation disclosed herein are described in context of an aortic valve, however, the prosthetic valves and methods can be adapted for use in other heart valves (such as, for example, in a pulmonary valve, a tricuspid valve, or a mitral valve).

### Examples of the Disclosed Technology

FIG. 1 shows a prosthetic heart valve 100 (also referred to as a “prosthetic valve”), according to an example. The prosthetic valve 100 can include a stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

The valvular structure 104 can comprise a plurality of leaflets 112 (for example, three leaflets, as shown in FIG. 1), collectively forming a leaflet structure, which can be arranged to collapse in a tricuspid arrangement. The leaflets 112 can be secured to one another at their adjacent sides (for example, commissure tabs) to form commissures 114 of the valvular structure 104. For example, each leaflet 112 can comprise opposing commissure tabs disposed on opposite sides of the leaflet 112 and a cusp edge portion extending between the opposing commissure tabs. The cusp edge portion of the leaflets 112 can have an undulating, curved scalloped shape, and can be secured directly to the frame 102 (for example, by sutures). However, in alternate examples, the cusp edge portion of the leaflets 112 can be secured to an inner skirt which is then secured to the frame 102. In some examples, the leaflets 112 can be formed of pericardial tissue (for example, bovine pericardial tissue), biocompatible synthetic materials, or various other suitable natural or synthetic materials as known in the art and described in U.S. Patent No. 6,730,118, which is incorporated by reference herein.

In some examples, the outer skirt 106 can be an annular skirt. In some examples, the outer skirt 106 can comprise one or more skirt portions that are connected together and/or individually connected to the frame 102. In some examples, instead of having a relatively straight upper edge portion, as shown in FIG. 1, the outer skirt 106 can have an undulating upper edge portion that extends along and is secured to the angled struts 134. Examples of such outer skirts, as well as various other outer skirts, that can be used with the frame 102 can be found in PCT Application No.US2023/025210 filed June 13, 2023, which is incorporated by reference herein.

The outer skirt 106 (and/or an inner skirt, discussed below, of the prosthetic valve 100) can be wholly or partly formed of any suitable biological material, synthetic material (for example, any of various polymers), or combinations thereof. In some examples, the skirt(s) can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirt(s) can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt(s) can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirt(s) can comprise a sponge material or foam, such as polyurethane foam. In some examples, the skirt(s) can comprise natural tissue, such as pericardium (for example, bovine pericardium, porcine pericardium, equine pericardium, or pericardium from other sources).

The frame 102 can be radially compressible and expandable between a radially compressed (or collapsed) configuration (for example, similar to the compressed state of the prosthetic valve 250 shown in FIG. 3 and discussed below) and a radially expanded configuration (the expanded configuration is shown in FIGS. 1 and 2). In FIG. 2, the frame 102 is shown alone (without an outer skirt and valve structure) for illustrative purposes.

The frame 102 can be made of any of various suitable plastically-expandable materials (for example, stainless steel, etc.) or self-expanding materials (for example, Nitinol). When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the prosthetic valve can be advanced from the delivery sheath, which allows the valve to expand to its functional size.

Suitable plastically-expandable materials that can be used to form the frame 102 include, without limitation, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIG. 2, the frame 102 can comprise a plurality of interconnected struts 116 which form multiple rows of open cells 118 between the outflow end 110 and the inflow end 108 of the frame 102. In some examples, the frame 102 can comprise three rows of cells 118 with a first (upper in the orientation shown in FIG. 2) row of cells 120 disposed at the outflow end 110. The first row of cells 120 comprises cells 118 that are elongated in an axial direction (relative to a central longitudinal axis 122 of the frame 102), as compared to cells 118 in the remaining rows of cells. For example, the cells 118 of the first row of cells 120 can have a longer axial length than cells 118 in other rows of cells, which can include a second row of cells 126 and a third row of cells 128, the third row of cells 128 disposed at the inflow end 108 and the second row of cells 126 disposed between the first row of cells 120 and the third row of cells 128.

In some examples, as shown in FIG. 2, each row of cells can include nine cells 118. In such examples, the frame 102 can be referred to as a nine-cell frame.

In alternate examples, the frame 102 can comprise more than three rows of cells (for example, four or five) and/or more or less than nine cells per row. In some examples, the cells 118 in the first row of cells 120 may not be elongated compared to cells 118 in the remaining rows of cells of the frame 102 (the second row of cells 126 and the third row of cells 128).

The interconnected struts 116 can include a plurality of angled struts 130, 132, 134, and 136 arranged in a plurality of rows of circumferentially extending rows of angled struts, with the rows being arranged along the length of the frame 102 between the outflow end 110 and the inflow end 108. For example, the frame 102 can comprise a first row of angled struts 130 arranged end-to-end and extending circumferentially at the inflow end 108 of the frame; a second row of circumferentially extending, angled struts 132; a third row of circumferentially extending, angled struts 134; and a fourth row of circumferentially extending, angled struts 136 at the outflow end 110 of the frame 102. The fourth row of angled struts 136 can be connected to the third row of angled struts 134 by a plurality of axially extending window struts 138 (or window strut portions) and a plurality of axial (or axially extending) struts 140. The axially extending window struts 138 (which can also be referred to as “axial struts that include a commissure window”) define commissure windows 142 (for example, open windows) that are spaced apart from one another around the frame 102, in a circumferential direction, and which are adapted to receive a pair of commissure tabs of a pair of adjacent leaflets arranged into a commissure (for example, the commissure 114 of a pair of adjacent leaflets 112 shown in FIG. 1). In some examples, the commissure windows 142 and/or the axially extending window struts 138 defining the commissure windows 142 can be referred to herein as commissure features or commissure supports, each commissure feature or support configured to receive and/or be secured to a pair of commissure tabs of a pair of adjacent leaflets.

One or more axial struts 140 (for example, two axial struts, as shown in FIG. 2) can be positioned between, in the circumferential direction, two commissure windows 142 formed by the window struts 138. Since the frame 102 can include fewer cells per row (for example, nine) and fewer axial struts 140 between each commissure window 142, as compared to other (for example, traditional) prosthetic heart valves, each cell 118 can have an increased width (in the circumferential direction), thereby providing a larger opening for blood flow and/or coronary access.

Each axial strut 140 and each window strut 138 can extend from a location defined by the convergence of the lower ends (for example, ends arranged inward of and farthest away from the outflow end 110) of two angled struts 136 (which can also be referred to as an “upper strut junction” or an “upper elongated strut junction”) to another location defined by the convergence of the upper ends (for example, ends arranged closer to the outflow end 110) of two angled struts 134 (which can also be referred to as a “lower strut junction” or a “lower elongate strut junction”). Each axial strut 140 and each window strut 138 can form an axial side of two adjacent cells of the first row of cells 120.

In some examples, each axial strut 140 can have a width that is larger than a width of the angled struts 130, 132, 134, and 136. As used herein, a “width” of a strut is measured between opposing locations on opposing surfaces of a strut that extend between the radially facing inner and outer surfaces of the strut (relative to the central longitudinal axis 122 of the frame 102). A “thickness” of a strut is measured between opposing locations on the radially facing inner and outer surfaces of a strut and is perpendicular to the width of the strut. In some examples, the width of the axial struts 140 is 50-200%, 75-150%, or at least 100% larger than (for example, double) the width of the angled struts of the frame 102.

By providing the axial struts 140 with a width that is greater than the width of other, angled struts of the frame 102, a larger contact area is provided for when the leaflets 112 contact the wider axial struts 140 during systole, thereby distributing the stress and reducing the extent to which the leaflets 112 may fold over the axial struts 140, radially outward through the cells 118. As a result, a long-term durability of the leaflets 112 can be increased.

Since the cells 118 of the frame 102 can have a relatively large width of the opening compared to alternate prosthetic valves that have more than nine cells per row (as discussed above), the wider axial struts 140 can be more easily incorporated into the frame 102, without sacrificing open space for blood flow and/or coronary access.

Returning to FIG. 1, commissure tabs 115 of adjacent leaflets 112 can be secured together to form commissures 114. Each commissure 114 of the prosthetic heart valve 100 can comprise two commissure tabs 115 paired together, one from each of two adjacent leaflets 112, and extending through a commissure window 142 of the frame 102. Each commissure 114 can be secured to the window struts 138 forming the commissure window 142.

The cusp edge portion (for example, scallop edge) of each leaflet 112 can be secured to the frame 102 via one or more fasteners (for example, sutures). In some examples, the cusp edge portion of each leaflet 112 can be secured directly to the struts of the frame 102 (for example, angled struts 130, 132, and 134). For example, the cusp edge portions of the leaflets 112 can be sutured to the angled struts 130, 132, and 134 that generally follow the contour of the cusp edge portions of the leaflets 112.

In some examples, the cusp edge portion of the leaflets 112 can be secured to an inner skirt (not shown) and the inner skirt can then be secured directly to the frame 102.

Various methods for securing the leaflets 112 to a frame, such as the frame 102, are disclosed in PCT Application No. US2022/049666, filed November 11, 2022, and U.S. Provisional Patent Application No. 63/300,302, filed January 18, 2022, both of which are incorporated by reference herein.

As shown in FIG. 2, in some examples, one or more of or each of the axial struts 140 can comprise an inflow end portion 146 (for example, an end portion that is closest to the inflow end 108) and an outflow end portion 148 that are widened relative to a middle portion 150 of the axial strut 140. In some examples, the inflow end portion 146 of the axial strut 140 can comprise an aperture 147. The apertures 147 can be configured to receive fasteners (for example, sutures) for attaching soft components of the prosthetic heart valve 100 to the frame 102. For example, the outer skirt 106 can be positioned around the outer surface of the frame 102 and an upper or outflow edge portion of the outer skirt 106 can be secured to the apertures 147 by fasteners 149 (for example, sutures), as shown in FIG. 1.

The interconnected struts 116 can also comprise horizontal struts 182 that extend between adjacent cells 118 of a row of cells of the frame 102 (FIG. 2). The horizontal struts 182 can extend in a circumferential direction and also be referred to as circumferentially extending struts 182. The horizontal struts 182 can connect angled struts of two adjacent rows of angled struts of the frame 102 to one another. For example, each horizontal strut 182 can connect to two angled struts of one row of struts (for example, two of the struts 134) and two angled struts in another, adjacent row of struts (for example, two of the struts 132). As a result, an angled strut extending between an axially extending window strut 138 and the horizontal strut 182 and an angled strut extending between the horizontal strut 182 and another horizontal strut 182 disposed adjacent to the inflow end 108 of the frame 102 can be aligned along an angled line that can follow a scallop line of the leaflets (when the leaflets are attached to the frame 102). Thus, the horizontal struts 182 can allow the angled struts to follow a shape that more closely matches a shape of the scallop line of the leaflets when the frame 102 is in the radially expanded configuration (as shown in FIGS. 1 and 2). Additionally, the horizontal struts 182 can serve as spacers that can maintain a specified gap between the angled struts when the frame 102 is in the radially compressed configuration, thereby reducing a risk of pinching the leaflets between the struts in the radially compressed configuration.

The frame 102 can further comprise a plurality of apex regions 152 formed at the inflow end 108 and the outflow end 110, each apex region 152 extending and forming a junction between two angled struts 130 at the inflow end 108 or two angled struts 136 at the outflow end 110. As such, the apex regions 152 are spaced apart from one another, in a circumferential direction at the inflow end 108 and the outflow end 110.

Each apex region 152 can comprise an apex (a highest or most outward extending, in an axial direction, point) and two thinned (or narrowed) strut portions extending from either side of the apex to a corresponding, wider angled strut 136 (at the outflow end 110) or to a corresponding, wider angled strut 130 (at the inflow end 108). Thus, each of the apex regions 152 at the outflow end 110 can form a narrowed transition region between and relative to the two angled struts 136 extending from the corresponding apex region 152 and each of the apex regions 152 at the inflow end 108 can form a narrowed transition region between and relative to the two angled struts 130 extending from the corresponding apex region 152. The thinned strut portions of the apex regions 152 can have a width that is smaller than a width of the angled struts 130 or 136.

Each apex region 152 and two corresponding angled struts 136 at the outflow end 110 can form an outflow strut 166 and each apex region 152 and two corresponding angled struts 130 at the inflow end 108 can form an inflow strut 168. Each outflow strut 166 and inflow strut 168 can have a length that includes an apex region 152 and the two angled struts 136 or 130 (or strut portions), respectively, on either side of the apex region 152. In some examples, each of the apex region 152 can form an angle between the two angled struts 130 or 136 extending from either side of the corresponding apex region 152. For example, an angle between the two angled struts 130 or 136 can be in a range of 120 to 140 degrees.

In some examples, each apex region 152 can comprise a curved, axially facing outer surface 172 and an arcuate or curved, axially facing inner depression 174 which forms the thinned strut portions. For example, the curved inner depression 174 can depress toward the curved outer surface 172 from an inner surface of the angled strut portions, thereby forming the (smaller width) thinned strut portions. Thus, the curved inner depressions 174 can be formed on a cell side of the apex region 152 (for example, as opposed to the outside of the apex region 152).

In some examples, the curved outer surface 172 of each apex region 152 can form a single, continuous curve from one angled strut portion on a first side of the apex region 152 to another angled strut portion on an opposite, second side of the apex region 152 (for example, the curved outer surface 172 can have a constant curvature). In some examples, a height of the apex regions 152 can be relatively small and contribute relatively little to the overall axial height of the radially expanded frame 102. Thus, the leaflets 112 secured to the frame 102 (FIG. 1) can be disposed close to the inflow end 108, thereby leaving a larger open space at the outflow end 110 of the frame 102 that is not blocked by the leaflets 112.

In some examples, gaps through which blood can pass through the frame 102 can be defined between inflow ends of the junctions 196 and the outflow edge of the outer skirt 106 (for example, the open spaces in the cells). Downstream ends of the third row of angled struts 134 connect to each other at the junctions 196. A portion the junctions 196 connect to and/or form upstream (lower) ends of the axially extending window struts 138.

When commissures 114 are secured to the window struts 138, upstream edges of the commissures 114 are located at or adjacent to respective junctions 196 of the angled struts 134.

Additional details and examples of frames for prosthetic heart valves that include apex regions can be found in PCT Application No. PCT/US2022/025687 which is incorporated by reference herein.

In some examples, the prosthetic valves disclosed herein can be adapted for implantation in a native aortic valve. In some examples, the prosthetic valves disclosed herein can be adapted to be implanted in the other native valves of the heart (the pulmonary, mitral, and tricuspid valves). In some examples, the disclosed prosthetic valves can be implanted within a previously implanted prosthetic valve (which can be a prosthetic surgical valve or a prosthetic transcatheter heart valve) in a valve-in-valve procedure.

As discussed above, when constructed of a plastically-expandable material, the frame 102 (and thus the valve 100) can be crimped to a radially compressed state on a delivery catheter and then expanded inside a patient by an inflatable balloon or equivalent expansion mechanism. FIG. 3 shows a delivery apparatus 200, according to an example, that can be used to implant an expandable prosthetic heart valve (for example, the prosthetic heart valve 100 and/or other prosthetic heart valves described herein). In some examples, the delivery apparatus 200 is adapted for use in introducing a prosthetic valve into a heart.

The delivery apparatus 200 in the illustrated example of FIG. 3 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to move (for example, translate) longitudinally, along a central longitudinal axis 220 of the delivery apparatus 200, relative to one another to facilitate delivery and positioning of a prosthetic valve at an implantation site in a patient’s body (such as, for example, the implantation sites discussed above).

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222 (as shown in FIG. 3), or to an alternate component at the distal end of the delivery apparatus 200 (for example, a distal shoulder). An intermediate portion of the balloon 218 can overlay a valve mounting portion 224 of a distal end portion of the delivery apparatus 200 and a distal end portion of the balloon 218 can overlay a distal shoulder 226 of the delivery apparatus 200. The valve mounting portion 224 and the intermediate portion of the balloon 218 can be configured to receive a prosthetic heart valve in a radially compressed state. For example, as shown schematically in FIG. 3, a prosthetic heart valve 250 (which can be one of the prosthetic valves described herein) can be mounted in a radially compressed configuration around the balloon 218, at the valve mounting portion 224 of the delivery apparatus 200.

The balloon shoulder assembly, including the distal shoulder 226, is configured to maintain the prosthetic heart valve 250 (or other medical device) at a fixed position on the balloon 218 during delivery through the patient’s vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 250 is mounted in the radially compressed state on the valve mounting portion 224 and during delivery of the prosthetic valve to the target implantation site. As such, the distal tip portion 228 can be configured to resist movement of the prosthetic valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 250 at an implantation site.

An inner lumen of the inner shaft can be configured to receive a guidewire therethrough, for navigating the distal end portion of the delivery apparatus 200 to the target implantation site.

In some examples, the handle 202 can include a steering mechanism configured to adjust the curvature of the distal end portion of the delivery apparatus 200. For example, as illustrated in FIG. 3, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Patent No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (for example, for fine positioning at the implantation site). Further details on the delivery apparatus 200 can be found in PCT Application No. PCT/US2021/047056, which is incorporated by reference herein.

In other examples, when constructed of a self-expandable material, a prosthetic valve (such as the valve 100 and/or other prosthetic valves disclosed herein) can be crimped to a radially compressed state and restrained in the compressed state by insertion into a sheath or equivalent mechanism of a delivery catheter. Once inside the body, the prosthetic valve can be advanced from the delivery sheath, which allows the valve to expand to its functional size. Exemplary delivery apparatus for use with a self-expandable valve that can be utilized with the prosthetic valve and methods disclosed herein are described in U.S. Patent Application No. 17/934,524, which is incorporated by reference herein.

As introduced above, exemplary prosthetic heart valves and methods of prosthetic valve implantation disclosed herein can prevent migration of a prosthetic heart valve and limit or prevent a force from being exerted on the annulus by the prosthetic heart valve when in a radially expanded state.

FIG. 4 illustrates an exemplary prosthetic valve 300 and implantation thereof within a native aortic valve 350. Specifically, FIG. 4 shows a distal end portion of an exemplary delivery apparatus 310 with the prosthetic valve 300 crimped thereon in a radially compressed state (also referred to as the radially compressed prosthetic valve 300a). In some examples, the prosthetic valve 300 can have one or more of the features described above with respect to the prosthetic valves 100, 250. In some examples, the prosthetic valve 300 is the prosthetic valve 100 and includes a frame 102. In some examples, the prosthetic valve 300 can have one or more features that differ from the prosthetic valve 100, 250. In some examples, the delivery apparatus 310 can have one or more of the features described above with respect to the delivery apparatus 200. In some examples, the delivery apparatus 310 can have one or more features that differ from the delivery apparatus 200.

As shown in FIG. 4, in the present examples, the prosthetic valve 300a can be a balloon-expandable valve and can be crimped onto an inner shaft 311 over an inflatable balloon 312 between a distal shoulder 314 and a proximal shoulder 316. The shoulders 314 and 316 can be positioned around the shaft 311 within distal and proximal portions of the balloon 312. In some examples, when the prosthetic valve 300a is crimped onto the delivery apparatus 310, the shoulders 314, 316 can protect or shield the distal and proximal ends of the prosthetic valve 300a to help prevent the ends of the valve from snagging on a catheter wall, a vessel wall, or other structure during transcatheter delivery of the prosthetic valve 300a. In some examples, the shoulders 314, 316 can also limit or prevent axial migration of the prosthetic valve 300a relative to the inner shaft 311. In some examples, crimping the distal end and/or the proximal end of the prosthetic valve 300a to a small outer diameter than the rest of the valve can also limit or prevent snagging or axial migration of the prosthetic valve 300a during transcatheter delivery thereof. When the prosthetic valve 300a is delivered to an implantation location (such as, for example, at the native aortic valve 350), the balloon 312 can be inflated to radially expand the prosthetic valve 300 for its deployment and implantation.

In some examples, a method of implanting the prosthetic valve 300 can include inserting a distal end portion of the delivery apparatus 310 having the radially compressed prosthetic valve 300a mounted thereon into vasculature of a patient, advancing the distal end portion of the delivery apparatus 310 through the vasculature (for example, via one or more of the delivery techniques discussed below) until the radially compressed prosthetic valve 300a is within the native aortic valve 350, aligning the radially compressed prosthetic valve 300a relative to one or more anatomical features of the native aortic valve 350, and radially expanding the prosthetic valve 300 to a radially expanded state (also referred to as the radially expanded prosthetic valve 300b) within the native aortic valve 350, such that an inflow end 302 of the radially expanded prosthetic valve 300b is above an annulus 352 of the aortic valve 350 with an inflow end portion of the prosthetic valve between the native leaflets 356 of the aortic valve 350 and an outflow end 304 of the radially expanded prosthetic valve 300b is below a sinotubular junction (STJ) 354.

In some examples, the method can include, prior to inserting the distal end portion of the delivery apparatus into the vasculature of the patient, determining a distance between the annulus and the STJ of the patient (via, for example, x-ray, coronary angiogram, fluoroscopy, or other imaging techniques) and selecting a prosthetic heart valve that has a height in its radially expanded state that is less than the distance between the annulus and the STJ.

As can be seen in FIG. 4, the radially compressed prosthetic valve 300a can be axially elongated and can have a greater length relative to when the radially expanded prosthetic valve 300b. When radially expanded (that is, when transitioned from the radially compressed prosthetic valve 300a to the radially expanded prosthetic valve 300b), the prosthetic valve 300 is axially foreshortened to a shorter length relative to the radially compressed prosthetic valve 300a. In some examples, aligning the compressed prosthetic valve 300a relative to the one or more anatomical features of the native aortic valve 350 can include positioning the inflow end 302 below the annulus 352 and/or positioning the outflow end 304 above the STJ 354. In this manner, in some examples, when the prosthetic valve foreshortens upon radial expansion, the inflow end 302 moves to a location above the annulus 352 and the outflow end 304 moves to a location below the STJ 354.

In some examples, the radially compressed prosthetic valve 300a has a length a, and the radially expanded prosthetic valve 300b has a length b. In some examples, the length b is less than the length a. In some examples, the length b is less than a distance c between the annulus 352 and the STJ 354. In some examples, the length a can be greater than the distance c. In other examples the length a can be less than the distance c. In some examples, the length a is in a range of 20 mm to 87 mm, such as a range of 20 mm to 35 mm, a range of 30 mm to 70 mm, a range of 40 mm to 60 mm, a range of 45 mm to 55 mm, etc. In some examples, the length b is in a range of 10 mm to 29 mm, such as a range of 12 mm to 26 mm, a range of 15.5 mm to 22.5 mm, a range of 18 mm to 20 mm, etc. In some examples, a prosthetic valve 300b has an expanded working diameter of about 20 mm and a length b of about 15.5 mm to about 18.5 mm. In some examples, a prosthetic valve 300b has an expanded working diameter of about 23 mm and a length b of about 18 mm to about 21 mm. In some examples, a prosthetic valve 300b has an expanded working diameter of about 26 mm and a length b of about 20 mm to about 23 mm. In some examples, a prosthetic valve 300b has an expanded working diameter of about 29 mm and a length b of about 22.5 mm to about 25.5 mm. In some examples, the distance c is in a range of 15 mm to 30 mm, depending on the anatomy of the patient.

The length b being less than the distance c can enable the radially expanded prosthetic valve 300b to be seated (implanted) within the aortic valve 350 without extending into or below the annulus 352 or into or above the STJ 354. For example, in the radially expanded state, native leaflets 356 of the aortic valve 350 can extend along an exterior surface 306 of the prosthetic valve 300b. The inflow end 302 of the radially expanded prosthetic valve 300b can abut the native leaflets 356 above the annulus 352 (for example, abut a base portion of the leaflets 356 just above the annulus 352). The outflow end 304 of the radially expanded prosthetic valve 300b can abut a portion of a top wall 360 of the aortic root 351, which is below the STJ 354.

Accordingly, in the example shown in FIG. 4, the radially expanded prosthetic valve 300b can be captured (or otherwise retained) between the annulus 352 and the STJ 354 and the entirety of the prosthetic valve 300b can be located within the aortic root 351. For example, upstream migration of the prosthetic valve 300b into a left ventricle 362 can be prevented (or otherwise limited) via the contact between the inflow end 302 and the native leaflet tissue 356 above the aortic annulus 352 and downstream migration of the prosthetic valve 300b into an ascending aorta 364 can be prevented (or otherwise limited) via the contact between the outflow end 304 and a portion 360 of the aortic wall 358 below the STJ 354. Additionally, as the radially expanded prosthetic valve 300b is seated above the annulus 352, an amount of force exerted on the annular tissue can be reduced relative to an expanded prosthetic valve that is seated within and/or extends below the annulus. Positioning and anchoring the prosthetic valve 300b in this manner is particularly advantageous for replacing an insufficient native aortic valve. Moreover, if the prosthetic valve 300 is undersized, such as if the patient has extensive leaflet or commissural calcification, the outer skirt of the prosthetic valve is positioned relatively higher up in the aortic root and can seal against the native leaflets, which typically forms a narrower orifice than the native annulus.

In some examples, a prosthetic valve can additionally include one or more anti-migration features and a method of implanting the prosthetic valve can include (in addition to the steps described above), deploying the one or more anti-migration features. For example, FIG. 5 illustrates an exemplary prosthetic valve 400 and implantation thereof within a native aortic valve 350. Specifically, FIG. 5 shows the distal end portion of the delivery apparatus 310 with the prosthetic valve 400 crimped thereon in a radially compressed state (also referred to as the radially compressed prosthetic valve 400a). The prosthetic valve 400 is the same as and implanted in the same manner as the prosthetic valve 300 except for the additional features disclosed below.

The prosthetic valve 400 can additionally include one or more anti-migration features. For example, the prosthetic valve 400 can include one or more frame anchors or anchor members 408, such as in the form of clipping members. The frame anchors 408 can be coupled to a main body of a frame of the prosthetic valve 400 (for example, frame 102) at locations axially spaced from the inflow end 403 of the valve. The frame anchors 408 can be configured to engage the native leaflets 356 of the aortic valve 350 (for example, at or adjacent the native commissures) to assist in retaining a position of the prosthetic heart valve 400b relative to the aortic annulus 352.

In some examples, the frame anchors 408 may be evenly distributed around a circumference of the frame of the prosthetic valve 400b. In some examples, each frame anchor 408 may be coupled to the frame of the prosthetic valve 400b. For example, the frame anchors 408 can be formed from of the same material as the frame 102, and may be integrally formed therewith. For example, the frame and the frame anchors can be formed (for example, laser cut) from the same piece of material (for example, a piece of Nitinol). In another example, each frame anchor and the frame can be separately formed and the frame anchors can be subsequently joined thereto. Various techniques and mechanisms can be used to connect the frame anchors to the frame, such as welding the frame anchors to the main body, adhesively bonding the frame anchor to the main body, or using mechanical connectors, such as sutures, screws, rivets, pins, or various other connection means.

In some examples, each frame anchor 408 can be coupled to the frame 102 such that an outer skirt of the prosthetic valve 400 extends at least partially between each frame anchor and the main body. In particular, the outer skirt may include holes, cutouts, openings, apertures, etc. at the locations at which each frame anchor 408 is attached to the main body. In some examples, each frame anchor 408 may be at least partially covered, or substantially covered by (for example, wrapped in) a protective covering that protects the native leaflets from directly contacting the frame anchor 408 and can minimize or prevent trauma to the patient’s native tissues by avoiding direct contact between a metal anchor and the tissue by avoiding direct contact between a metal anchor and the tissue. In some examples, the protective covering may be formed from the same material as the inner and/or outer skirt of the prosthetic valve 400. In other examples, the frame anchors 408 can be uncovered.

As shown in FIG. 5, in a deployed configuration, the frame anchors 408 can include one or more legs or hooks that extend toward the inflow end 403 of the prosthetic valve 400b and capture the leaflets 356 between the exterior surface 406 of the prosthetic valve 400b and the interior surface of the frame anchors 408. In some examples, the frame anchors 408 can bend or deform when transitioning from a delivery configuration to a deployed configuration (illustrated in FIG. 5). For example, the frame anchors 408 can be formed from a shape-memory material, such as Nitinol. When formed from a shape-memory material, the anchors 408 can be shape set in the deployed configuration. Thus, when the radially compressed prosthetic valve 400a is mounted onto a delivery apparatus for delivery into a patient’s body, the frame anchors 408 are deformed to the delivery configuration and retained in the delivery configuration by retaining elements (for example, tethers or a sleeve) of the delivery apparatus. When released from the retaining elements at or near the implantation site, the anchor members 408 can automatically revert to the deployed configuration under their own resiliency and capture the leaflets 356.

In the deployed configuration, each frame anchor 408 can be placed around or can capture the free edges of one or more of the native leaflets 356 (for example, around the free edges of a pair of the native leaflets at or in close proximity to a native commissure or at locations between the commissures of the native leaflets 356). In some examples, each frame anchor 408 can capture or clamp a native leaflet between the frame anchor 408 and the exterior surface of the frame 102. In this manner, the anchors function as “clipping members” by clipping the native leaflets 356 against the frame. In other examples, the frame anchors 408 do not press the native leaflets 356 against the frame but extend over the free edge of a native leaflet. In either case, when the prosthetic heart valve 400b is implanted in the aortic valve 350, the frame anchors 408 can assist in retaining the prosthetic valve 400b within the native aortic valve 350 by resisting migration of the prosthetic valve 400b in an upstream direction (toward the left ventricle 362). In some examples, the frame anchors 408 can additionally assist in resisting migration of the prosthetic valve 400b in a downstream direction (toward the ascending aorta 364) when the native leaflets are clamped between the frame anchors 408 and the exterior surface of the frame 102.

In some examples, as illustrated in FIG. 4, the frame anchors 408 can extend from the exterior surface 406 of the radially expanded prosthetic valve 400b (at a central portion 410 of the valve 400b) and downward toward the inflow end 403. In some examples, the frame anchors 408 can have a length that enables capture of the free edge portions of the leaflets 356. In some examples, the frame anchors 408 can extend from the exterior surface 406 of the radially expanded prosthetic valve 400b at an outflow end portion (that is, a portion of the radially expanded prosthetic valve prosthetic valve 400b closer to the outflow end 404 than the inflow end 403) and can have a longer length (relative to the illustrated example) that enables capture of the leaflets 356.

Additional description of the frame anchors and other clipping members (such as, retaining arms) that can be utilized with the prosthetic valves disclosed herein are disclosed in PCT Application No. US2023/028328 and U.S. Patent Application Publication No. 2016/0228240, which are each incorporated by reference herein. In some examples, a frame anchor can take the form of a stent or annular support member that is deployed behind the native leaflets 356 (radially outside of the native leaflets), such that the native leaflets are captured between the support member and the frame of the prosthetic valve (such as frame 102). Examples of such support members are disclosed in U.S. Patent Application Publication No. 2016/0228240.

Although not shown in FIGS. 4 and 5, prosthetic valves 300, 400 can include an outer skirt or sealing member, such as outer skirt 106, to establish a seal against the native leaflets 356 when the prosthetic valve is implanted. In lieu or in addition to an outer skirt 106, a prosthetic valve can include other sealing features or mechanisms. For example, the prosthetic valve 400 can include an outer skirt 412. The outer skirt 412 can be coupled to a frame 102 of the prosthetic valve 400 on the exterior surface 406 of the valve near the inflow end 403.

In some examples, the outer skirt 412 can have one or more of the features discussed above with respect to the outer skirt 106. In some examples, the outer skirt 412 can be used in combination with another outer skirt, such as the outer skirt 106. In some examples, the outer skirt 412 can be used in lieu of the outer skirt 106. In some examples, the outer skirt 412 can have a thickness that is greater than that of the outer skirt 106, and can thereby form an annular flange 414 on around the exterior surface 406 of the radially expanded prosthetic valve 400b. In some examples, the skirt 412 is an extension of the skirt 106.

In some examples, the outer skirt 412 can comprise a fabric body formed from a plurality of strands or yarns that are woven, knitted, or otherwise secured together. In some examples, the outer skirt 412 can comprise a woven or knitted fabric comprising a fabric base layer and a plurality of pile yarns (for example, loop yarn) or floating yarns that extend radially outward and away from the exterior surface 406. The pile yarns or floating yarns can give the outer skirt 412 a fuzzy, plush, or velour-like texture. Further, the yarns can be relatively densely packed together and configured to create the annular flange 414 that projects outwardly from the exterior surface 406 of the valve 400b. The annular flange 414 can be configured to contact and/or engage the native leaflets 356 (for example, contacting and engaging tissue at a base of the leaflets near the aortic annulus 352) to assist in retaining the radially expanded prosthetic heart valve 400b in position relative to the aortic annulus 352.

Thus, when the prosthetic heart valve 400b is implanted in the aortic valve 350, the annular flange 414 can assist in establishing a seal with the native leaflets 356 to prevent or minimize paravalvular leakage (PVL). The flange 414 can encourage tissue ingrowth, which over time can assist in anchoring the prosthetic valve in place against the native leaflets.

In some examples, the outer skirt 412 can additionally include an annular extension 416 that extends into or below the aortic annulus 352. Nonetheless, the inflow end 403 of the frame 102 of the prosthetic valve 400 is positioned above the annulus 352, and desirably seats on or abuts a base portion of the native leaflets 356. The outflow end 404 of the prosthetic valve (which also corresponds to the outflow end of the frame 102) abuts a portion of a top wall 360 of the aortic root 351 below the STJ 354. Thus, in examples that include a skirt that extends into the annulus 352, all metal components (including frame 102 and any frame anchors 408) are located entirely within the aortic root 351 above the annulus 352 and below the STJ 354.

In some examples, the extension 416 can be relatively thinner than the flange 414. In some examples, the skirt extension 416 can comprise a polymeric material. In some examples, the polymeric material can be relatively thromboresistant or have a thromboresistance that is greater than that of a fabric. In some examples, the polymeric material can be relatively non-abrasive or smooth. For example, in some instances, the polymeric material can be TPU. In other examples, the skirt extension 416 can comprise a different relatively thromboresistant material such as polytetrafluoroethylene (PTFE). In this way, tissue ingrowth is less likely to occur along the extension 416.

In other examples, the skirt extension 416 can comprises a fabric (including any of the fabrics already disclosed herein) to encourage tissue ingrowth along the skirt extension 416 to assist in anchoring the prosthetic valve to the native annulus 352.

In some examples, the prosthetic valve 400 includes an outer skirt 106 (instead of a skirt 414 forming a flange 416) and the extension 416 is an extension of the skirt 106.

It should be noted that the additional features disclosed in FIG. 5 are optional features that can be combined with each other in any combination. For example, the prosthetic valve 400 can include one or more frame anchors 406 but not skirt 414 or extension 416. In another example, the prosthetic valve 400 can include skirt 414 and/or extension 416 but not any frame anchors 406.

Further details on skirts for prosthetic heart valves that comprise a knitted or woven material and pile or floating yarns and/or a thinner layer extending from the inflow end of the prosthetic valve are disclosed in U.S. Patent Application Publication No. 2019/0374337, International Patent Publication No. WO 2021/202636, and PCT Application No. US2023/025210, which are each incorporated by reference herein.

The prosthetic valve 100 is particularly suited for implantation in a supra-annular position as described herein. For example, as shown in FIG. 1, the prosthetic valve 100 has relatively large openings between the free edges of the leaflets 112 (the upper edges of the leaflets in FIG. 1) and the angled struts 116 defining the outflow end 110 of the frame 102. These relatively large openings permit access to the coronary arteries with a catheter via the openings in the frame in the event such access is required in a post-implantation procedure. Moreover, the relatively large openings can ensure adequate blood flow to the coronary arteries during normal operating cycles of the prosthetic valve.

Another benefit of the prosthetic valve 100 is that the frame 102 has outflow apices and inflow apices 152 that are relatively smooth and flat compared to known frames that include crown like structures forming apices at the opposite ends of the frame. The relatively flat outflow apices 152 are desirable because they can avoid trauma to the inner surface of the aortic root. Similarly, the relatively flat inflow apices 152 can avoid trauma to the native leaflets 356.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A method of implanting a prosthetic heart valve, the method comprising: inserting a distal end portion of a delivery apparatus having the prosthetic heart valve mounted thereon in a radially compressed state into a vasculature of a patient; advancing the distal end portion of the delivery apparatus through the vasculature until the radially compressed prosthetic heart valve is within an aortic root of the patient; aligning the radially compressed prosthetic heart valve relative to one or more anatomical features of the aortic root; and radially expanding the prosthetic heart valve to a radially expanded state within the aortic root, wherein an inflow end of a frame of the radially expanded prosthetic heart valve is above an annulus of an aortic valve and an outflow end of the frame of the radially expanded prosthetic heart valve is below a sinotubular junction (STJ) of the aortic root.

Example 2. The method of any example disclosed herein, particularly example 1, wherein the aligning the radially compressed prosthetic heart valve relative to the one or more anatomical features of the aortic root comprises positioning the inflow end of the frame of the prosthetic heart valve below the annulus.

Example 3. The method of any example disclosed herein, particularly example 1 or example 2, wherein the aligning the radially compressed prosthetic heart valve relative to the one or more anatomical features of the aortic root comprises positioning the outflow end of the frame of the prosthetic heart valve above the STJ.

Example 4. The method of any example disclosed herein, particularly examples 1-3, wherein, in the radially expanded state, native leaflets of the aortic valve extend along an exterior surface of the prosthetic heart valve.

Example 5. The method of any example disclosed herein, particularly example 4, wherein, in the radially expanded state, a portion of the prosthetic valve corresponding to the inflow end of the frame contacts the native leaflets above the annulus.

Example 6. The method of any example disclosed herein, particularly example 5, wherein, in the radially expanded state, the portion of the prosthetic valve corresponding to the inflow end of the frame abuts a base portion of the native leaflets.

Example 7. The method of any example disclosed herein, particularly examples 1-6, wherein, in the radially expanded state, a portion of the prosthetic valve corresponding to the outflow end of the frame abuts a portion of a top wall of the aortic root below the STJ.

Example 8. The method of any example disclosed herein, particularly examples 1-7, wherein a height of the frame of the prosthetic heart valve is less than a distance between the annulus and the STJ.

Example 9. The method of any example disclosed herein, particularly example 8, wherein the height of the frame of the prosthetic heart valve is in a range of 15.5 mm to 22.5 mm.

Example 10. The method of any example disclosed herein, particularly examples 1-9, further comprising deploying one or more anti-migration features of the prosthetic heart valve.

Example 11. The method of any example disclosed herein, particularly example 10, wherein the deploying one or more anti-migration features comprises deploying one or more frame anchors over a native leaflet free edge.

Example 12. The method of any example disclosed herein, particularly examples 1-11, further comprising engaging an annular flange of an outer skirt of the prosthetic heart valve with native tissue above the annulus of the aortic valve.

Example 13. The method of any example disclosed herein, particularly examples 1-12, wherein the prosthetic heart valve further comprises an ingrowth-resistant annular extension that extends into the annulus when the prosthetic heart valve is in the radially expanded state within the aortic root.

Example 14. A method of treating an aortic valve of a patient, the method comprising: determining a distance between an annulus and an STJ of the aortic root of the patient; selecting a prosthetic heart valve that has a height in a radially expanded state that is less than the distance between the annulus and the STJ; inserting a distal end portion of a delivery apparatus having the prosthetic heart valve mounted thereon in a radially compressed state into a vasculature of the patient; advancing the distal end portion of the delivery apparatus through the vasculature until the radially compressed prosthetic heart valve is between native leaflets of the aortic valve of the patient; and radially expanding the prosthetic heart valve to the radially expanded state within the aortic root, wherein an inflow end of the radially expanded prosthetic heart valve is above the annulus and an outflow end of the radially expanded prosthetic heart valve is below the STJ.

Example 15. The method of any example disclosed herein, particularly example 14, aligning the radially compressed prosthetic heart valve relative to one or more anatomical features of the aortic root.

Example 16. The method of any example disclosed herein, particularly example 15, wherein the aligning the radially compressed prosthetic heart valve relative to the one or more anatomical features of the aortic root comprises positioning the inflow end of the prosthetic heart valve below the annulus.

Example 17. The method of any example disclosed herein, particularly example 15 or example 16, wherein the aligning the radially compressed prosthetic heart valve relative to the one or more anatomical features of the aortic root comprises positioning the outflow end of the prosthetic heart valve above the STJ.

Example 18. The method of any example disclosed herein, particularly examples 14-17, wherein, in the radially expanded state, the native leaflets of the aortic valve extend along an exterior surface of the prosthetic heart valve.

Example 19. The method of any example disclosed herein, particularly example 18, wherein, in the radially expanded state, the inflow end of the prosthetic heart valve contacts the native leaflets above the annulus.

Example 20. The method of any example disclosed herein, particularly example 18 or example 19, wherein, in the radially expanded state, the inflow end of the prosthetic heart valve abuts a base portion of the native leaflets.

Example 21. The method of any example disclosed herein, particularly examples 14-20, wherein, in the radially expanded state, the outflow end of the prosthetic heart valve abuts a portion of a top wall of the aortic root below the STJ.

Example 22. The method of any example disclosed herein, particularly examples 14-21, wherein a height of the prosthetic heart valve is in a range of 15.5 mm to 22.5 mm.

Example 23. The method of any example disclosed herein, particularly examples 14-22, further comprising deploying one or more anti-migration features of the prosthetic heart valve.

Example 24. The method of any example disclosed herein, particularly example 23, wherein the deploying one or more anti-migration features comprises deploying one or more clipping members over a native leaflet free edge.

Example 25. The method of any example disclosed herein, particularly examples 14-24, further comprising engaging an annular flange of an outer skirt of the prosthetic heart valve with native tissue above the annulus of the aortic valve.

Example 26. A method of treating a heart on a simulation, the method comprising: inserting a distal end portion of a delivery apparatus having a prosthetic heart valve mounted thereon in a radially compressed state into a vasculature on the simulation; advancing the distal end portion of the delivery apparatus through the vasculature until the radially compressed prosthetic heart valve is within an aortic root of the heart on the simulation; aligning the radially compressed prosthetic heart valve relative to one or more anatomical features of the aortic root; and radially expanding the prosthetic heart valve to a radially expanded state within the aortic root, wherein an inflow end of the radially expanded prosthetic heart valve is above an annulus of an aortic valve and an outflow end of the radially expanded prosthetic heart valve is below a sinotubular junction (STJ) of the aortic root.

Example 27. The method of any example disclosed herein, particularly example 26, wherein, in the radially expanded state, native leaflets of the aortic valve extend along an exterior surface of the prosthetic heart valve.

Example 28. The method of any example disclosed herein, particularly example 27, wherein, in the radially expanded state, the inflow end of the prosthetic heart valve contacts the native leaflets above the annulus.

Example 29. The method of any example disclosed herein, particularly example 28, wherein, in the radially expanded state, the inflow end of the prosthetic heart valve abuts a base portion of the native leaflets.

Example 30. The method of any example disclosed herein, particularly examples 26-29, wherein, in the radially expanded state, the outflow end of the prosthetic heart valve abuts a portion of a top wall of the aortic root below the STJ.

Example 31. The method of any example disclosed herein, particularly examples 26-30, wherein a height of the prosthetic heart valve is less than a distance between the annulus and the STJ.

Example 32. A method of implanting a radially compressible and expandable prosthetic heart valve, the method comprising: radially expanding the prosthetic heart valve from a radially compressed state to a radially expanded state within an aortic root, wherein an inflow end of a frame of the radially expanded prosthetic heart valve is above an annulus of an aortic valve and an outflow end of the frame of the radially expanded prosthetic heart valve is below a sinotubular junction (STJ) of the aortic valve.

Example 33. The method of any example disclosed herein, particularly example 32, wherein an entirety of the frame of the prosthetic heart valve is within the aortic root upstream of the annulus of the aortic valve and downstream of the STJ after the prosthetic heart valve is radially expanded.

Example 34. The method of any example disclosed herein, particularly example 32 or example 33, wherein the prosthetic heart valve comprises a skirt that extends upstream of the inflow end of the frame and into the annulus of the aortic valve.

Example 35. The method of any example disclosed herein, particularly example 32, wherein an entirety of the prosthetic heart valve is within the aortic root upstream of the annulus of the aortic valve and downstream of the STJ after the prosthetic heart valve is expanded.

Example 36. A method of implanting a radially compressible and expandable prosthetic heart valve, the method comprising a step for: radially expanding the prosthetic heart valve from a radially compressed state to a radially expanded state within an aortic root, wherein an inflow end of the radially expanded prosthetic heart valve is above an annulus of the aortic valve and an outflow end of the radially expanded prosthetic heart valve is below a sinotubular junction (STJ) of the aortic valve.

Example 37. A method of implanting a radially compressible and expandable prosthetic heart valve including the steps of any example disclosed herein, particularly examples 1-36, wherein the steps are performed on a living animal or on a simulation, such as on a cadaver, cadaver heart, anthropomorphic ghost, simulator (for example, with body parts, heart, tissue, etc. being simulated).

The features described herein with regard to any example can be combined with other features described in any one or more of the other examples, unless otherwise stated. For example, any one or more of the features of one prosthetic heart valve can be combined with any one or more features of another prosthetic heart valve. As another example, any one or more features or steps of one method can be combined with any one or more features or steps of another method.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A method of implanting a prosthetic heart valve, the method comprising:
inserting a distal end portion of a delivery apparatus having the prosthetic heart valve mounted thereon in a radially compressed state into a vasculature of a patient;
advancing the distal end portion of the delivery apparatus through the vasculature until the radially compressed prosthetic heart valve is within an aortic root of the patient;
aligning the radially compressed prosthetic heart valve relative to one or more anatomical features of the aortic root; and
radially expanding the prosthetic heart valve to a radially expanded state within the aortic root, wherein an inflow end of a frame of the radially expanded prosthetic heart valve is above an annulus of an aortic valve and an outflow end of the frame of the radially expanded prosthetic heart valve is below a sinotubular junction (STJ) of the aortic root.

2. The method of claim 1, wherein the aligning the radially compressed prosthetic heart valve relative to the one or more anatomical features of the aortic root comprises positioning the inflow end of the frame of the prosthetic heart valve below the annulus.

3. The method of claim 1, wherein the aligning the radially compressed prosthetic heart valve relative to the one or more anatomical features of the aortic root comprises positioning the outflow end of the frame of the prosthetic heart valve above the STJ.

4. The method of claim 1, wherein, in the radially expanded state, native leaflets of the aortic valve extend along an exterior surface of the prosthetic heart valve.

5. The method of claim 4, wherein, in the radially expanded state, a portion of the prosthetic valve corresponding to the inflow end of the frame contacts the native leaflets above the annulus.

6. The method of claim 5, wherein, in the radially expanded state, the portion of the prosthetic valve corresponding to the inflow end of the frame abuts a base portion of the native leaflets.

7. The method of claim 1, wherein, in the radially expanded state, a portion of the prosthetic valve corresponding to the outflow end of the frame abuts a portion of a top wall of the aortic root below the STJ.

8. The method of claim 1, wherein a height of the frame of the prosthetic heart valve is less than a distance between the annulus and the STJ.

9. The method of claim 8, wherein the height of the frame of the prosthetic heart valve is in a range of 15.5 mm to 22.5 mm.

10. The method of claim 1, further comprising deploying one or more anti-migration features of the prosthetic heart valve.

11. The method of claim 10, wherein the deploying one or more anti-migration features comprises deploying one or more frame anchors over a native leaflet free edge.

12. The method of claim 1, further comprising engaging an annular flange of an outer skirt of the prosthetic heart valve with native tissue above the annulus of the aortic valve.

13. The method of claim 1, wherein the prosthetic heart valve further comprises an ingrowth-resistant annular extension that extends into the annulus when the prosthetic heart valve is in the radially expanded state within the aortic root.

14. A method of treating an aortic valve of a patient, the method comprising:
determining a distance between an annulus and an STJ of an aortic root of the patient;
selecting a prosthetic heart valve that has a height in a radially expanded state that is less than the distance between the annulus and the STJ;
inserting a distal end portion of a delivery apparatus having the prosthetic heart valve mounted thereon in a radially compressed state into a vasculature of the patient;
advancing the distal end portion of the delivery apparatus through the vasculature until the radially compressed prosthetic heart valve is between native leaflets of the aortic valve of the patient; and
radially expanding the prosthetic heart valve to the radially expanded state within the aortic root, wherein an inflow end of the radially expanded prosthetic heart valve is above the annulus and an outflow end of the radially expanded prosthetic heart valve is below the STJ.

15. The method of claim 14, aligning the radially compressed prosthetic heart valve relative to one or more anatomical features by positioning the inflow end of the prosthetic heart valve below the annulus and positioning the outflow end of the prosthetic heart valve above the STJ.

16. The method of claim 14, wherein a height of the prosthetic heart valve is in a range of 15.5 mm to 22.5 mm.

17. A method of implanting a radially compressible and expandable prosthetic heart valve, the method comprising:
radially expanding the prosthetic heart valve from a radially compressed state to a radially expanded state within an aortic root, wherein an inflow end of a frame of the radially expanded prosthetic heart valve is above an annulus of an aortic valve and an outflow end of the frame of the radially expanded prosthetic heart valve is below a sinotubular junction (STJ) of the aortic valve.

18. The method of claim 17, wherein an entirety of the frame of the prosthetic heart valve is within the aortic root upstream of the annulus of the aortic valve and downstream of the STJ after the prosthetic heart valve is radially expanded.

19. The method of either of claim 17, wherein the prosthetic heart valve comprises a skirt that extends upstream of the inflow end of the frame and into the annulus of the aortic valve.

20. The method of claim 17, wherein an entirety of the prosthetic heart valve is within the aortic root upstream of the annulus of the aortic valve and downstream of the STJ after the prosthetic heart valve is radially expanded.
